(19) United States
(12) Patent Application Publication
(10) Pub. No.: US 2025/0082513 A1
(43) Pub. Date: Mar. 13, 2025

(54) CRYOTHERAPY DRESSING SYSTEM

(71) Applicant: Todd R. Wurth, Brentwood, TN (US)

(72) Inventor: Todd R. Wurth, Brentwood, TN (US)

(21) Appl. No.: 18/955,203

(22) Filed: Nov. 21, 2024

Related U.S. Application Data
(63) Continuation-in-part of application No. 18/241,958, filed on Sep. 4, 2023.
Application No. 18/241,958 is a continuation of application No. 16/898,245, filed on Jun. 10, 2020, now Pat. No. 11,744,742.
(60) Provisional application No. 62/859,224, filed on Jun. 10, 2019.

(51) Int. Cl. A61F 13/05; A61F 13/04; A61M 1/00

(52) U.S. Cl. CPC A61F 13/05; A61F 13/046; A61M 1/74; A61M 1/962; A61M 2205/3606

(57) ABSTRACT

A dressing system for healing skin or a wound site on a subject comprises a dressing having a reservoir for holding a liquid and at least one light-emitting diode. The dressing may be configured for placement proximate to a postoperative surgical wound site of the subject or on the facial area of the subject. The dressing system includes a cooling chamber external to the dressing configured to cool the liquid. The dressing system includes a pump configured to circulate the liquid between the cooling chamber and the reservoir. The dressing system includes a cooled liquid hose for circulating the cooled liquid between the cooling chamber and the reservoir of the dressing.

### CROSS-REFERENCE TO RELATED APPLICATIONS

This application is a continuation-in-part of and claims the benefit of U.S. patent application Ser. No. 18/241,958, filed Sep. 4, 2023 and currently pending, which is a continuation of and claims the benefit of U.S. patent application Ser. No. 16/898,245, now U.S. Pat. No. 11,744,742, filed Jun. 10, 2020, which claims the benefit of U.S. Provisional Patent Application No. 62/859,224, filed Jun. 10, 2019. The entire disclosures, including the specifications and drawings, of all above-referenced applications are incorporated herein by reference.

### FIELD OF THE INVENTION

The present disclosure relates to a dressing system. More specifically, the present invention is generally directed towards a dressing system that incorporates cryotherapy component and a light-emitting diode therapy component.

### BACKGROUND

Wound care is a critical aspect of healing and surgery. Indeed, in 2016, the global wound care market was valued at approximately $23.3 billion and is estimated to significantly grow. While promoting fast and complete wound care is important on the one hand, these considerations must be weighed against and balanced with subject comfort and pain management. Subject pain management is especially relevant in the present clinical environment of minimizing prescribed narcotic use. What are needed, then, are systems and methods for effectively promoting healing in wounds that are comfortable for the subject and reduce subject postoperative pain.

### SUMMARY OF THE INVENTION

In one aspect, a dressing system for cooling skin or a wound site on a subject is disclosed. The dressing system comprises a dressing having a reservoir. The dressing may be configured for placement proximate to a postoperative surgical wound site of the subject. The postoperative surgical wound site may be, for example, a postoperative splint, within a postoperative dressing, within cast padding under a cast. The dressing may comprise a supplemental dressing configured for use with a primary dressing. The dressing may be constructed of a pliable material.

The dressing system comprises a cooling chamber external to the dressing. A thermoelectric unit or similar device may be disposed adjacent the cooling chamber and configured to decrease or change the temperature of the liquid in the cooling chamber.

The dressing may comprise an upper component and a lower component, where the upper component and lower component may be selectively detachable from one another through the use of coupling components. The lower component may be configured for placement proximate the skin or wound site of a subject, and the upper component may be placed on and connected to the lower component when the dressing is in use. The upper component may be configured to be reusable, while the lower component may be configured as a consumable or disposable component.

The upper component may include a reservoir layer configured to receive and hold a liquid in the reservoir. The upper component may include a light-emitting diode layer comprised of at least one light-emitting diode. In some aspects, the at least one light-emitting diode may be a plurality of light emitting diodes. The light emitting diode(s) may radiate a particular wavelength and is configured such that the radiation is directed in the direction of the wound site in order to promote healing in the subject and an anti-bacteria environment in and around the wound site.

The dressing system comprises a pump configured to circulate a liquid between the cooling chamber and the reservoir of the dressing. The pump may be configured to move liquid from the cooling chamber to the reservoir through a coolant hose and the liquid from the reservoir to the cooling chamber through a return hose. The liquid may comprise water or saline and may be sterile.

The dressing system may include a suction element for removing wound drainage or sweat from the area proximate to the dressing. The dressing system may have a drain cavity for receiving and storing the wound drainage or sweat and a drain hose in fluid communication with the suction element for moving the wound drainage or sweat from the suction element to the drain cavity. The pump may be configured to create the suction to the suction element through the drain hose. In some aspects, a second pump may be configured to create the suction to the suction element through the drain hose will the first pump may be used to circulate the liquid.

The dressing system may include a flush port component in the lower component to enable the delivery and extraction of liquids, gels, and/or medicative substances to and from the wound site to which the dressing is applied. The liquid, gel, or medicative substance may be delivered to the one or more ports by a delivery tube connected to the lower component. A second removal tube may be connected to the lower component for removing or extracting the liquid, gel, or medicative substance. A third pump may be provided to operate the flush port component and supply and remove the liquid, gel, and/or medicative substance by means of the delivery tube and removal tube.

In some aspects, the dressing may include conductive electrodes that interact with the wound site to promote wound healing. In other aspects, the dressing may include a combination of intersecting elastic bands proximate to the wound site to apply tension and compression and promote wound healing.

The lower component may include a temperature strip configured to detect the surface temperature of the skin of the subject to which the dressing is applied.

The lower component may include an activity alarm patch configured to measure and record physical activity or medical measurements at the wound site and provide an output that alerts or records events not in accordance with prescribed medical care instructions

The dressing system may include a control unit having a user interface displaying various controls and settings for modification and operation by a user. The control unit may include status indicators for the various controls and settings. The control unit may cause the pumps to operate when certain controls and/or settings are met or selected by a user.

The dressing system may comprise a body external to the dressing. One or more of the cooling chamber, the drain cavity, the thermoelectric unit, or the pump may be disposed on the body.

In another aspect, a method of promoting healing in a subject is disclosed. The method of promoting healing in the subject comprises circulating chilled liquid from the cooling chamber to the reservoir of the dressing and directing light-emitting diodes at the wound site, the dressing positioned at, or proximate to, skin of the subject. The skin of the subject may comprise a wound site or facial skin. The method may include applying negative pressure at, or proximate to, the dressing to remove wound drainage or sweat.

In yet another aspect, a method of rejuvenating facial skin is disclosed. The method comprises circulating chilled liquid from the cooling chamber to the reservoir of the dressing, the dressing positioned at, or proximate to, the facial skin of the subject. The method may include applying negative pressure at, or proximate to, the dressing.

Other and further objects of the invention, together with the features of novelty appurtenant thereto, will appear in the course of the following description.

### BRIEF DESCRIPTION OF THE SEVERAL VIEWS OF THE DRAWING

In the accompanying drawings, which form a part of the specification and are to be read in conjunction therewith in which like reference numerals are used to indicate like or similar parts in the various views:

FIG. 1 is a schematic view of an embodiment of the dressing system.

FIG. 2 is a schematic view of another embodiment of the dressing system.

FIG. 3 is a schematic view of yet another embodiment of the dressing system.

FIG. 4A illustrates a close-up bottom view of a dressing according to one embodiment of the dressing system.

FIG. 4B illustrates a close-up bottom view of a dressing according to another embodiment of the dressing system.

FIG. 5 is a schematic view of an embodiment of the dressing system.

FIG. 6. is a schematic view of another embodiment of the dressing system.

FIG. 7 illustrates a schematic view of an upper component of a dressing according to one embodiment of the dressing system.

FIG. 8 illustrates a schematic view of a lower component of a dressing according to one embodiment of the dressing system.

FIG. 9 illustrates a schematic view of the lower component of the dressing according to one embodiment of the dressing system.

### DESCRIPTION OF THE INVENTION

Reference now will be made in detail to the embodiments of the present disclosure. It will be apparent to those of ordinary skill in the art that various modifications and variations can be made to the teachings of the present disclosure without departing from the scope of the disclosure. For instance, features illustrated or described as part of one embodiment can be used with another embodiment to yield a further embodiment.

Thus, it is intended that the present disclosure covers such modifications and variations as come within the scope of the appended claims and their equivalents. Other objects, features, and aspects of the present disclosure are disclosed in or are apparent from the following description. It is to be understood by one of ordinary skill in the art that the present disclosure is a description of exemplary embodiments only and is not intended as limited to the broader aspects of the present disclosure.

For the sake of clarity, not all reference numerals are necessarily present in each drawing figure. In addition, positional terms such as “upper,” “lower,” “side,” “top,” “bottom,” “vertical,” “horizontal,” etc. refer to the system when in the orientation shown in the drawings. The skilled artisan will recognize that the system can assume different orientations when in use.

Systems, and methods of using, dressing systems have been developed. The systems and methods are intended to allow convenient and safe cooling, light therapy, and/or suction of a local site of a subject to, for example, promote wound healing or rejuvenate skin.

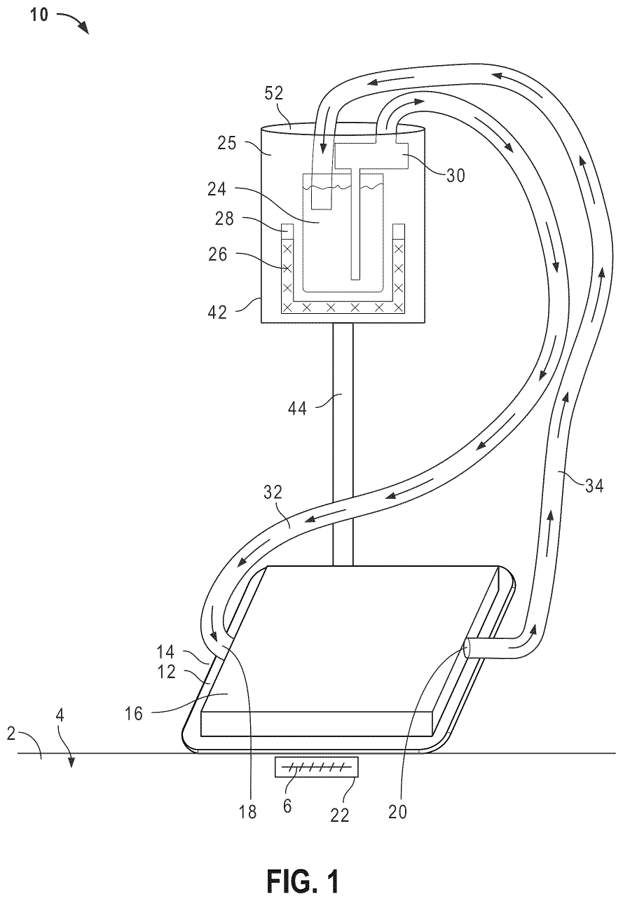

Referring now to the dressing system 10 of FIGS. 1-5, a dressing system 10 for cooling skin 4 or a wound site 6 on a subject 2 includes a dressing 12. The subject 2 may be any animal, including a human. The dressing 12 may comprise a removable dressing cover 14 disposed around the dressing 12. The removable dressing cover 14 may be removed or replaced, for example, between uses and between subjects, such as for cleaning of the dressing cover 14 or the dressing 12. The dressing 12 and the dressing cover 14 may each be constructed of any suitable material, such as a fabric (e.g., cotton or polyester), a polymer such as PVC, polyethylene, polyurethane, or latex.

The dressing 12 includes a reservoir 16, or a bladder, configured to receive and hold a liquid. The reservoir 16 may include a reservoir inlet 18 and a reservoir outlet 20 for receiving the liquid into and out, respectively, of the reservoir 16. The reservoir inlet 18 and the reservoir outlet 20 may each be in fluid communication with the reservoir such that the liquid may move between the inlet 18 and the outlet 20 and the reservoir 16. The reservoir 16 may be sealed and impermeable to the liquid except for the reservoir inlet 18 and the reservoir outlet 20. The reservoir 16 may also be impermeable to external liquids except as through the reservoir inlet 18 and the reservoir outlet 20. Advantageously, sealed reservoir 16 prevents the liquid from escaping onto the subject 2 when the dressing 12 is in use on the subject 2, and creates a “closed” dressing system 10. The reservoir 16 may have walls constructed of any suitable material, such as a polymer (e.g., Low Density Polyethylene (LDPE)).

The dressing 12 may be configured for placement proximate to the postoperative site 6, such as a postoperative surgical wound site, such as a postoperative splint, within a postoperative dressing, within cast padding under a cast, of the subject 2. In some embodiments, the dressing 12 is a supplemental dressing 12 for use with a primary dressing 22 that is in direct contact with the subject 2, and the dressing 12 (and/or the dressing cover 14) is in indirect contact with the subject 2 in at least one area, if not the across the entire dressing 12 (and/or dressing cover 14). The dressing 12, and the dressing cover 14, may be elastic, flexible, and/or self-adhesive such that it may be used on the subject 2, for example, within cast padding worn by the subject 2.

The dressing system 10 comprises a cooling chamber 24 external to the dressing 12. The cooling chamber 24 may be disposed within a cooling chamber body 25, the body 25 constructed of any suitable material. “External” means that the cooling chamber is a discrete from, and not included on, the dressing 12. In some embodiments, “external” refers to the cooling chamber 24 not being worn by the subject. A coolant 26, such as ice or a pre-cooled refrigerant pack, may be in contact (whether directly or indirectly) with the cooling chamber 24 to cool the liquid within the chamber 24. The coolant 26 may be disposed, for example, around at least part of the cooling chamber 24. The coolant 26 may be disposed within a coolant cavity 28. In some embodiments, a cooling unit 68, such as a thermoelectric cooler or a heat exchanger, may be used to cool the liquid within the chamber 24.

The dressing system 10 may comprise a pump 30. The pump may be configured to circulate the liquid between the cooling chamber 24 and the reservoir 16 of the dressing 12. The pump 30 may be configured to move the liquid from the cooling chamber 24 to the reservoir 16 through a coolant hose 32 and the liquid from the reservoir 16 to the cooling chamber 24 through a return hose 34 in a loop. The pump 30 may be operable continuously, intermittently, via a timer, or in variable flow rates (e.g., at least one flow speeds, at least two flow speeds, at least three flow speeds, etc.). In some embodiments, a thermometer 60 is disposed on the dressing 12 and is in communication with a control unit 62 configured to selectively operate the pump 30. The control unit 62 may cause the pump 30 to operate when an initial threshold temperature is met and read by the thermometer 60. By way of example, if the initial threshold temperature is 90 degrees F., once the control unit 62 reads the temperature being above 90 degrees, F, the control unit 62 could cause the pump 30 to operate, thereby circulating the liquid and cooling the skin 4. Once the thermometer reads a certain subsequent threshold temperature, such as 40 degrees F., the control unit 62 may cause the turn off the pump 30. The control unit 62 may cause the pump 30 to operate at variable speeds based on one or more threshold temperatures (e.g., a low pump speed at a cold threshold temperature, a moderate pump speed at a warm threshold temperature, a high pump speed at a hot threshold temperature). The control unit 62 may communicate with the pump 30 and the thermometer wiredly or wirelessly. The control unit 62 (along with pumps 30, 42 and cavity 38) may be disposed on or within any suitable component of the system 10, such as within the cooling chamber body 25. The control unit may operatively and selectively control the cooling unit 68. A power supply 64 (whether AC or DC, such as a battery) may power the pumps 30, 42, and the control unit 62, and the cooling unit 68.

The liquid may comprise, for example, refrigerant, water, or saline, and may be sterile or non-sterile. Non-sterile liquid may be advantageous for personal use where sterile conditions are not important or practicable. Sterile liquid may be advantageous for medical settings, such as in clinical care settings.

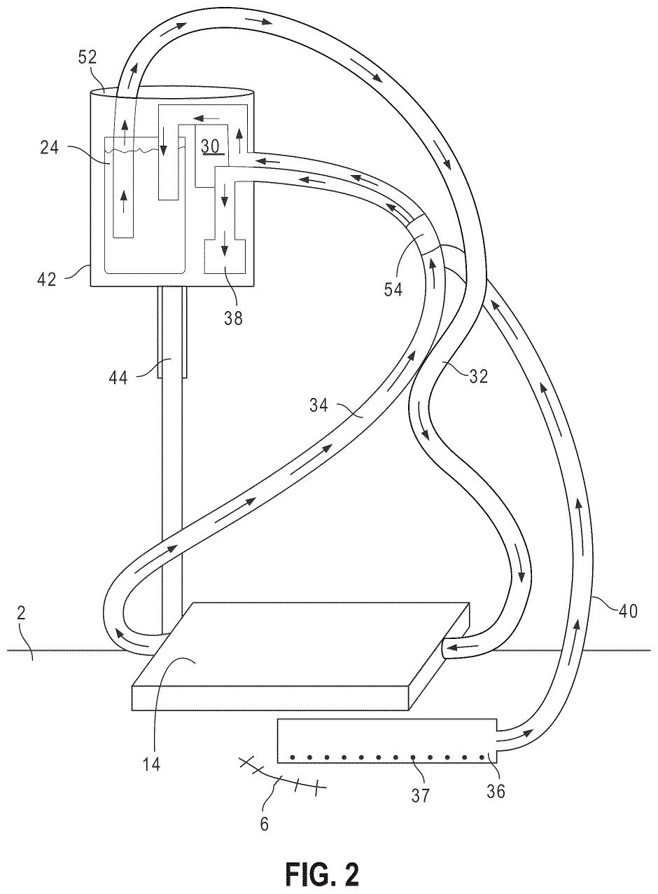

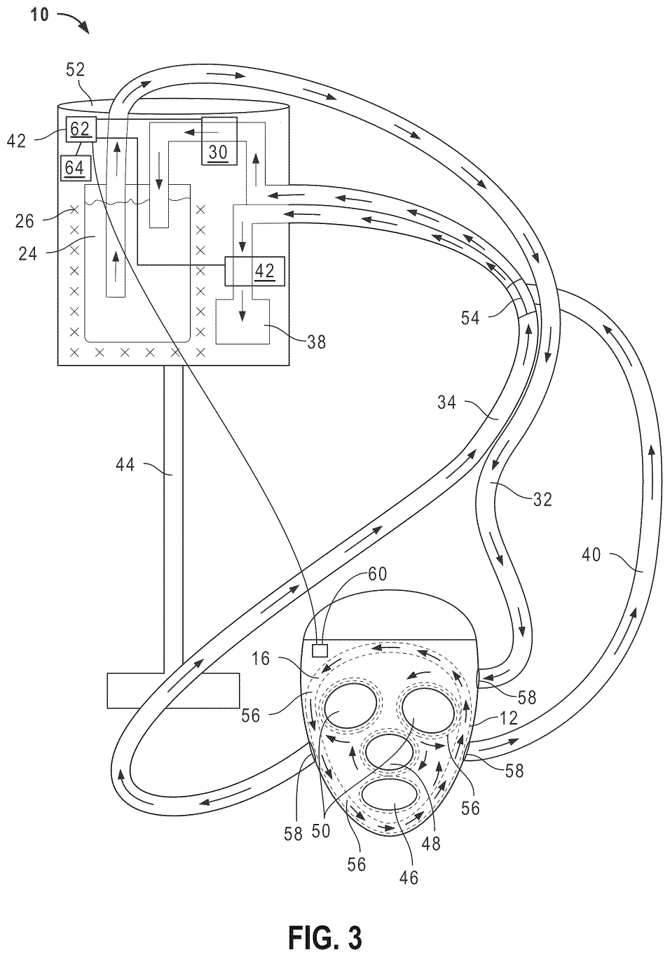

As shown in FIG. 2, the dressing system 10 may include a suction element 36 (e.g., a drain) for removing wound drainage or sweat from the area proximate to the dressing 10. The dressing system 10 may have a drain cavity 38 for receiving and storing the wound drainage or sweat and a drain hose 40 in fluid communication with the suction element 36 for moving the wound drainage or sweat from the suction element 36 to the drain cavity 38. A second pump 42 may be configured to create the suction to the suction element 36 through the drain hose 40. The suction element 36 may include one or more suction holes 37 through which suction is applied from the second pump 42 through the drain hose 40 to the area proximate to the dressing 12. The one or more suction holes 37 may be disposed in the dressing 12 or in the dressing 12 and the cover 14. In some embodiments, the single pump 30 is used to operate both the suction element 36 and circulate the liquid. The suction holes 37 may be positioned on the dressing 12 such that the suction holes 37 generally face the skin 4 of the subject 2 such that they pull drainage from the skin 4 of the subject (or dressing area surrounding the skin 4). The suction element 36 may be provided as a discrete element (as shown in FIG. 2) or as part of the dressing 12 (as shown in FIGS. 3-5).

In some embodiments, the suction pump 42 may be used to create suction with drain hose 40 and return hose 34 while the liquid pump 30 may be used to circulate the liquid. This multi-pump configuration may be beneficial to, for example, maintain separation of sweat and wound drainage from the liquid. In some embodiments, a valve 54 (e.g., a bivalve and/or a check valve) may be used to modulate, and separate, suction from the pump 42 to the drain hose 40 and/or the return hose 34, or to ensure a one-way flow through the hoses 32, 34, 40.

The dressing system 10 may comprise a body 43 external to the dressing 12. The body 43 may be, for example, disposed on a medical cart 44 (e.g., a medical stand), located proximate to the subject 2. One or more of the cooling chamber 24, the drain cavity 38, the coolant 26, or the pump 30 may be disposed on or in the body 43. The body 43 may be constructed of any suitable material, such as metal or a polymer (whether rigid or flexible). The body 43 being external to the dressing 12 is particularly advantageous, as it enables the dressing 12 to be compact and comfortable for the subject to wear 2. The coolant 26 may be replaced by, for example, removing and adding the coolant 26 by accessing the coolant 26 through a resealable open top 52 on the body 43. The top 52 may provide access to the coolant cavity 28 for replacing the coolant 26.

The dressing system 10 may comprise a body 42 external to the dressing 12. The body 42 may be, for example, disposed on a medical cart 44 (e.g., a medical stand), located proximate to the subject 2. One or more of the cooling chamber 24, the drain cavity 38, the coolant 26, or the pump 30 may be disposed on or in the body 42. The body 42 may be constructed of any suitable material, such as metal or a polymer (whether rigid or flexible). The body 42 being external to the dressing 12 is particularly advantageous, as it enables the dressing 12 to be compact and comfortable for the subject to wear 2. The coolant 26 may be replaced by, for example, removing and adding the coolant 26 by accessing the coolant 26 through a resealable open top 52 on the body 42. The top 52 may provide access to the coolant cavity 28 for replacing the coolant 26.

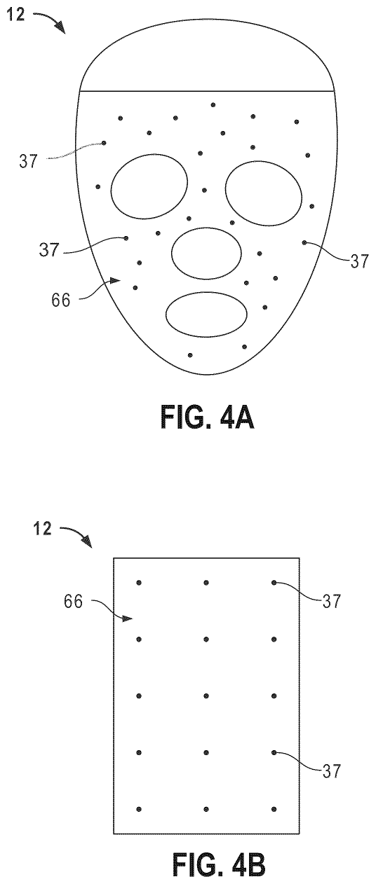

As shown in FIGS. 3 and 4A, the dressing 12 may be dimensioned and shaped to be cooperatively received on a face of the subject 2. The suction element 36 may be disposed on or in the dressing 12. Advantageously, when the dressing 12 (using suction of the suction element 36 and/or cooling of the circulating coolant 26) is used in the face of the subject 2, the dressing 12 promotes epidermal and dermal health and rejuvenation (e.g., a youthful appearance). The dressing 12 may have one or more of a mouth aperture 46, a nose aperture 48, or eye aperture(s) 50.

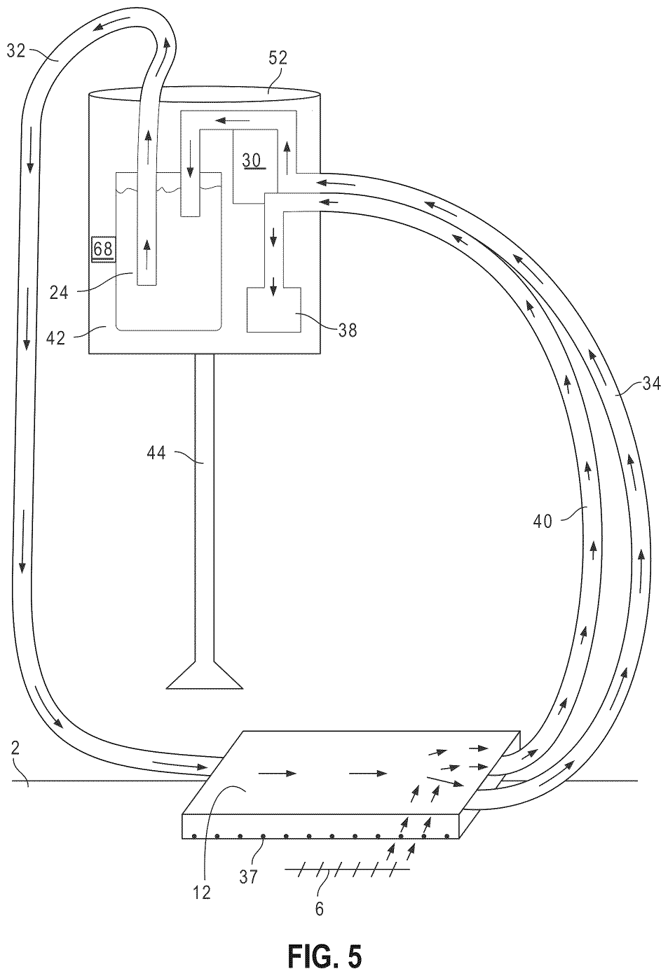

As shown in FIGS. 4-5, the dressing 12 may include the suction element 36, including the one or more suction holes 37 disposed on a skin surface 66 (i.e., the surface of the dressing 12 that faces or is in contact with the skin 4 or the wound site 6). The one or more suction holes 37 may be in fluid or gaseous communication with the pump 30, 42 through one or more internal channels (not shown) that to allow air and liquid movement to be pumped into the holes 37, through the drain hose 40, and into the drain cavity 38.

It is believed that the suction and/or cooling decreases blood flow to soft tissues, thereby lowering inflammation, lowering edema, limiting scaring, evacuating wound dressing, and promoting faster wound healing and recovery in postoperative applications (e.g., after facial cosmetic surgery). Moreover, the dressing 12 may have beneficial effects for facial skin care and rejuvenation, regardless of whether the subject 2 has a postoperative surgical wound.

As can be seen in FIG. 3, the dressing 12 may have a low profile design and be constructed of a soft and pliable material. This design is especially advantageous in facial applications, as eliminating or reducing sharp folds and having a profile that is shaped to contour a face prevents skin irritation. Using the features described herein, the dressing system 10 may be varied to include a dressing 12 that has a site-specific profile for use in, for example, plastic surgery, neurosurgery, urological surgery, ENT surgery, maxillofacial surgery, orthopedic surgery, and general surgery. That is, the dressing 12 may have a cooperative profile with the site-specific area on the subject 2 that is desired to be cooled and/or suctioned (e.g., neck, top or crown of head, nose, etc.) such that the dressing 12 can be worn on the subject 2 without folds or wrinkles in the dressing 12. This site-specific profile feature is believed to add to the healing and rejuvenating attributes of the dressing system 10.

Embodiments of the dressing system 10 described herein may be used in methods of promoting healing in a subject, such as promoting healing of the skin 4 of the subject. The method includes circulating the liquid from the cooling chamber 24 to the reservoir 16 of the dressing 12, the dressing 12 positioned at, or proximate to, the skin 4 of the subject 2. The skin 4 of the subject 2 may comprise a wound site 6, facial skin 4, or both. The method may include applying negative pressure at, or proximate to, the dressing 12, such as via suction element 36. The method may include suctioning and capturing wound drainage or sweat.

Embodiments of the dressing system 10 described herein may be used in methods of rejuvenating facial skin 4. The method includes circulating the liquid from the cooling chamber 24 to the reservoir 16 of the dressing 12, the dressing 12 positioned at, or proximate to, facial skin 4 of the subject 2. The method may include applying negative pressure at, or proximate to, the dressing 12, such as via suction element 36. The method may include suctioning and capturing wound drainage or sweat.

Embodiments of the dressing system 10 described herein may be used in methods of cooling and/or applying negative pressure to the skin 4 of the subject 2. The method includes circulating the liquid from the cooling chamber 24 to the reservoir 16 of the dressing 12, the dressing 12 positioned at, or proximate to, the skin 4 of the subject 2. The method may include applying negative pressure at, or proximate to, the dressing 12, such as via suction element 36. The method may include suctioning and capturing wound drainage or sweat. Also advantageously, the cooling and/or suction features of the present dressing system 10 reduce, or eliminate, postoperative pain.

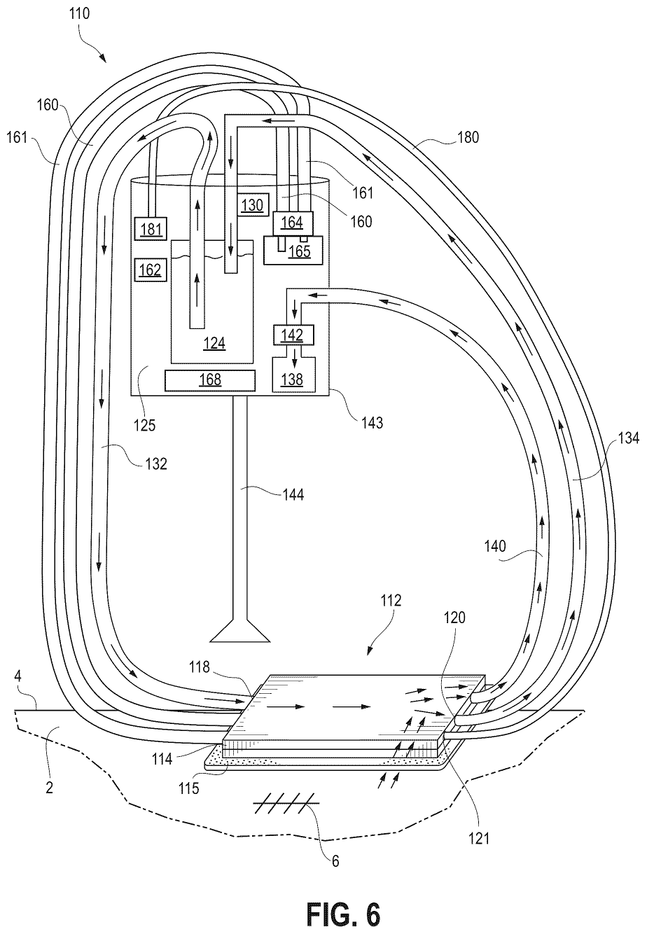

FIGS. 6-9 illustrate a dressing system 110 according to a second embodiment of the invention. Referring to FIGS. 6-9, a dressing system 110 for cooling skin 4 or a wound site 6 on a subject 2 includes a dressing 112. The dressing 112 may be configured for placement proximate to the postoperative site 6 of a subject 2, such as a postoperative surgical wound site, a postoperative splint, within a postoperative dressing, or within cast padding under a cast. The subject 2 may be any animal, including a human. The dressing 112 may comprise an upper component 114 and a lower component 115, where the upper component 114 and the lower component 115 may be selectively detachable from one another. The lower component 115 may be configured for placement proximate the skin 4 or wound site 6 of a subject 2 and the upper component 114 may be placed on and connected to the lower component 115 when the dressing 112 is in use.

The upper component 114 may be configured as a reusable component. All or a portion of the lower component 115 may be configured as a consumable component. At least a portion of the lower component 115 may be removed or replaced, for example, between uses and between subjects, such as for cleaning of the lower component 115. The upper component 114 and the lower component 115 may each be constructed of any suitable material, such as a fabric (e.g., cotton or polyester), a polymer such as PVC, polyethylene, polyurethane, or latex.

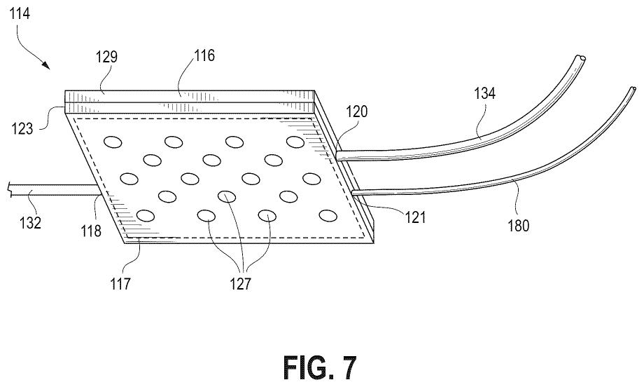

As can be seen in FIG. 7, the upper component 114 includes a reservoir layer 129, or a bladder, configured to receive and hold a liquid in a reservoir 116, a first coupling element 117, and a light-emitting diode (“LED”) layer 123. The reservoir layer 129 may include a reservoir inlet 118 and a reservoir outlet 120 for receiving the liquid into and out, respectively, of the reservoir 116. The reservoir inlet 118 and the reservoir outlet 120 may each be in fluid communication with the reservoir 116 such that the liquid may move between the inlet 118 and the outlet 120 and the reservoir 116. The reservoir 116 may be sealed and impermeable to the liquid except for the reservoir inlet 118 and the reservoir outlet 120. The reservoir layer 129 may also be impermeable to external liquids except as through the reservoir inlet 118 and the reservoir outlet 120. Advantageously, sealed reservoir layer 129 prevents the liquid from escaping onto the subject 2 when the dressing 112 is in use on the subject 2, and creates a “closed” dressing system 110. The reservoir layer 129 may have one or more walls constructed of any suitable material, such as a polymer (e.g., Low Density Polyethylene (LDPE)). The walls of the reservoir layer 129 may be configured such that the reservoir 116 contains internal passageways (not shown) for the liquid to circulate through while the dressing system 110 is in use on a subject 2. In some embodiments, the upper component 114 and/or the walls of the reservoir layer 129 may incorporate a thermally conductive film to facilitate equalizing thermal temperature differential and facilitate thermal equilibrium across the wound site 6. In some embodiments, the upper component 114 may include a thermally reflective film configured to insulate temperature differential.

The first coupling element 117 may be positioned on all or a portion of an outer surface of the upper component 114 and is configured to attach and detach the upper component 114 to the lower component 115. In some embodiments, the first coupling element 117 may be a plurality of coupling elements. The first coupling element 117 may be constructed of any suitable means for attaching and detaching the upper component 114 and lower component 115, including but not limited to a hook and loop (e.g., Velcro), laminate seal, adhesive strip, magnetic closures, snap buttons, etc.

The LED layer 123 is comprised of at least one LED 127, which receives power from a power connection 121. In some embodiments, the at least one LED 127 may be a plurality of LEDs. The LED(s) 127 may be any suitable size and shape and be configured in any suitable layout or arrangement. The power connection 121 may be located on a peripheral edge of the upper component 114, where power is drawn from an external power source 181 (e.g., a battery, an electrical outlet, or any AC or DC power source). An electrical cable or wire 180 may be connected to the power connection 121 to supply power to the LED layer 123 from the external power source 181. In some embodiments, the LED layer 123 may be configured such that it is positioned proximate to an exterior surface of the upper component 114, and the reservoir layer 129 is positioned under or behind the LED layer 123. In other embodiments, the LED layer 123 and the reservoir layer 129 may be configured such that the LED layer 123 is positioned under or behind the reservoir layer 129, where the LED layer 123 emits light through the reservoir layer 129 in the direction of the wound site 6. In such embodiments, the reservoir layer 129 may be configured from a transparent, translucent or semi-translucent material to allow light emitted from the LEDs 127 to pass through the reservoir layer 129.

The LED layer 123 radiates a particular wavelength and is configured such that the radiation is directed in the direction of the wound site 6. The purpose of the LED layer 123 is to promote healing in the subject 2 and also promotes an anti-bacteria environment in and around the wound site 6. The LED layer 123 operates independently from the cooling function of the reservoir 116. In some embodiments, the LED(s) 127 may emit a red wavelength, where the red wavelength may be a singular or full spectrum wavelength. The red wavelength may have a targeted range approximately between 630 and 830 nanometers (“nm”), depending on the desired depth of penetration. The purpose of the red wavelength is to improve cellular efficiency, reduce inflammation, and reduce pain associated with the wound site 6, where the wound site 6 may be the result of a post surgical wound resulting from both elective procedures (e.g., dermatological procedures) and non-elective procedures (e.g., general or orthopedic surgeries). In other embodiments, the LED(s) 127 may emit a blue wavelength, where the blue wavelength may be a singular or full spectrum wavelength. The blue wavelength may have a targeted range approximately between 405 and 470 nm, depending on the desired depth of penetration. The purpose of the blue wavelength is to reduce the risk of infection as a result of the antimicrobial effects of the blue wavelength.

The upper component 114 may be configured as a durable and reusable component such that the upper component 114 may be removed from use on a subject 2 and cleaned, sanitized and/or prepared for subsequent use on the same or a different subject 2. Alternatively, in some embodiments all or part of the upper component 114 may be configured as a consumable or one-time use component such that it is disposed of after use on a subject 2.

As shown in FIG. 6, the dressing system 110 may comprise a first pump 130. The pump 130 may be configured to circulate the liquid between the cooling chamber 124 and the reservoir layer 129 of the upper component 114. The pump 130 may be configured to move the liquid from the cooling chamber 124 to the reservoir layer 129 through a coolant hose 132 and the liquid from the reservoir layer 129 to the cooling chamber 124 through a return hose 134 in a loop. The pump 130 may be operable continuously, intermittently, via a timer, or in variable flow rates (e.g., at least one flow speeds, at least two flow speeds, at least three flow speeds, etc.).

The liquid may comprise, for example, refrigerant, water, or saline, and may be sterile or non-sterile. Non-sterile liquid may be advantageous for personal use where sterile conditions are not important or practicable. Sterile liquid may be advantageous for medical settings, such as in clinical care settings.

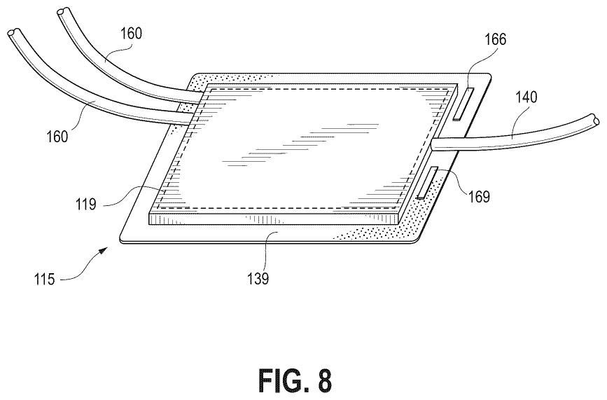

As can be seen in FIG. 8, the lower component 115 may include a second coupling element 119, a peripheral material 139, and a suction element 136. The second coupling element 119 may be positioned on all or a portion of an outer surface of the lower component 115 and configured to attach and detach the lower component 115 to and from the upper component 114. In some embodiments, the second coupling element 119 may be a plurality of coupling elements. The second coupling element 119 may be constructed of any suitable means for attaching and detaching the lower component 115 to and from the upper component 114, including but not limited to a hook and loop (e.g., Velcro), laminate seal, adhesive strip, magnetic closures, snap buttons, etc.

The upper component 114 and the lower component 115 may be electively attachable to one another at at least one attachment point formed by the first coupling element 117 and the second coupling element 119. In some embodiments, the first coupling element 117 removably connects to the second coupling element 119 to form a working hinge between the upper component 114 and the lower component 115. In some embodiments, the first coupling element 117 is disposed along one or more sides of the upper component 114 and the second coupling element 119 is disposed along one or more sides of the lower component 115 to allow the upper component 114 and lower component 115 to be removably attachable to one another. In some embodiments, the first coupling element 117 and the second coupling element 119 are disposed around the entire perimeters of the upper component 114 and lower component 115, respectively.

The peripheral material 139 may be positioned on an exterior surface of the lower component 115 and is configured to assist in removably adhering the lower component 115 to the skin 4 of a subject 2. In some embodiments, the peripheral material 139 may extend outwardly from the peripheral edges of the lower component 115 or may be one or more peripheral materials arranged on the exterior surface of the lower component 115.

The lower component 115, including the peripheral material 139, may be constructed of any suitable material such as a fabric (e.g., cotton or polyester) or a polymer such as PVC, polyethylene, polyurethane, or latex, and may include any suitable adhesive for removably adhering the lower component 115 to the skin 4 of a subject 2. Preferably, the lower component 115 and peripheral material 139 are constructed from a transparent, translucent or semi-translucent material to allow light emitted from the LEDs 127 of the upper component 114 to pass through the lower component 115 and to the skin 4 or wound site 6 of a subject 2 when dressing 112 is in use.

All or part of the lower component 115 may be configured as a consumable, disposable, or single-use component. In some embodiments, the entire lower component 115 is removable from the skin 4 of a subject 2 and then disposed of after use. In some embodiments, the peripheral material 139 is removably attached to the lower component 115 such that the peripheral material 139 may be detached from the lower component 115 and disposed of after a single use on a subject 2. The lower component 115 may be configured such that a new peripheral material 139 may be used for each new subject 2, where the upper component 114 and a portion of the lower component 115 of the dressing 112 may be reused on multiple subjects 2. In other embodiments, the peripheral material 139 may be used on multiple subjects 2.

As shown in FIGS. 6, 8, and 9, the lower component 115 of the dressing system 110 may include a negative pressure or suction element 136 (e.g., a drain) for removing wound drainage or sweat from the area proximate to the dressing 112. The dressing system 110 may have a drain cavity 138 for receiving and storing the wound drainage or sweat and a drain hose 140 in fluid communication with the suction element 136 for moving the wound drainage or sweat from the suction element 136 to the drain cavity 138. In some embodiments, the first pump 130 is used to operate both the suction element 136 and circulate the liquid in the reservoir 116. In other embodiments, a second pump 142 may be configured to create the suction to the suction element 136 through the drain hose 140 while the pump 130 may be used to circulate the liquid. This multi-pump configuration may be beneficial to, for example, maintain separation of sweat and wound drainage from the liquid. The pump 142 may be operable continuously, intermittently, via a timer, or in variable flow rates (e.g., at least one flow speeds, at least two flow speeds, at least three flow speeds, etc.). The suction element 136 may include one or more suction holes 137 through which suction is applied from the second pump 142 through the drain hose 140 to the area proximate to the dressing 112. The one or more suction holes 137 may be disposed in the lower component 115. The one or more suction holes 137 may be in fluid or gaseous communication with pump(s) 130, 142 through one or more internal channels (not shown) that to allow air and liquid movement to be pumped into the holes 137, through the drain hose 140, and into the drain cavity 138. In some embodiments, the lower component 115 is constructed from a porous, permeable or semi- permeable material (such as a synthetic sponge material) that enables the lower component 115 to absorb fluid or sweat from and transfer it away through the drain hose 140 by the suction element 136. The suction holes 137 may be positioned on the lower component 115 such that the suction holes 137 generally face the skin 4 of the subject 2 such that they pull drainage from the skin 4 of the subject (or dressing area surrounding the skin 4).

It is believed that the suction and/or cooling decreases blood flow to soft tissues, thereby lowering inflammation, lowering edema, limiting scaring, evacuating wound dressing, and promoting faster wound healing and recovery in postoperative applications (e.g., after facial cosmetic surgery). Moreover, the dressing 112 may have beneficial effects for facial skin care and rejuvenation, regardless of whether the subject 2 has a postoperative surgical wound.

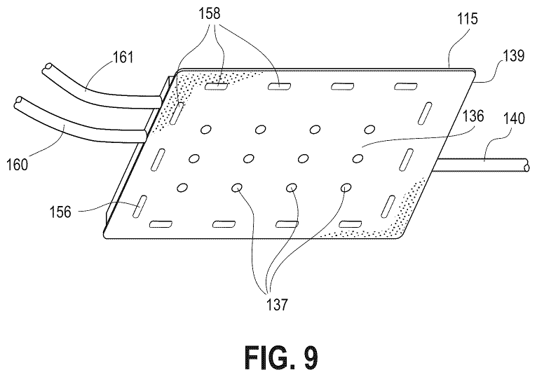

In some embodiments, the dressing 112 may include a flush port component 156 provided in the lower component 115. The flush port 156 may enable the delivery and extraction of liquids, gels and/or medicative substances to and from the wound site 6 to which the dressing 112 is applied. The flush port component 156 may include one or more ports 158 located on the underside of the lower component 115 (as shown in FIG. 9) for delivering a liquid, gel and/or medicative substance to the wound site 6. The liquid, gel and/or medicative substance may be delivered to the one or more ports 158 by a delivery tube 160 connected to the lower component 115 and in fluid communication with one or more ports 158. A second removal tube 161 may be connected to the lower component 115 for removing or extracting the liquid, gel and/or medicative substance from the wound site 6 after application. The second removal tube 161 may be in fluid communication with the one or more ports 158 for extracting the liquid, gel and/or medicative substance. In some embodiments, the delivery tube 160 may be used to both deliver the liquid, gel, and/or medicative substance to the wound site 6 and extract the liquid, gel, and/or medicative substance from the wound site 6 after application. The dressing system 110 may have a reservoir or container 165 for receiving and storing the liquid, gel, and/or medicative substance extracted after application through the removal tube 161. The reservoir 165 may also be configured to contain the liquid, gel, and/or medicative substance that is intended to be delivered to the dressing 112 through delivery tube 160 for application around the wound site 6. The removal tube 161 and/or the delivery tube 160 may be fluidly connected to the reservoir 165. A third pump 164 may be provided to operate the flush port component 156 and supply and remove the liquid, gel and/or medicative substance by means of the delivery tube 160 and removal tube 161. The pump 164 may be operable continuously, intermittently, via a timer, or in variable flow rates (e.g., at least one flow speeds, at least two flow speeds, at least three flow speeds, etc.). In some embodiments, the first pump 130 and/or the second pump 142 may be configured to operate the flush port component 156.

In some embodiments, the dressing 112 may include conductive electrodes that interact with the wound site 6 to promote wound healing. In other embodiments, the dressing 112 may include a combination of intersecting elastic bands proximate to the wound site 6 to apply tension and compression and promote wound healing (i.e., kinetic therapy).

The lower component 115 may include a temperature strip 166 integrated into the material of the lower component 115. The temperature strip 166 may be configured to detect the surface temperature of the skin 6 of the subject 2 to which the dressing 112 is applied. The temperature strip 166 may be connected to a control unit 162 (described below) by a wire or cable. The temperatures strip 166 may be configured to determine the temperature of the subject 2 and identify dynamic changes in temperature to enable monitoring of thermal activity and signal increased risk of infection at the wound site 6. The temperature strip 166 may be configured as a chromatic temperature film electrical sensor or other known temperature sensing component.

In some embodiments, the lower component 115 may include an activity alarm patch 169 that is provided on the peripheral material 139 of the lower component 115. The activity alarm patch 169 may be configured to measure and record physical activity or medical measurements at the wound site 6 and provide an output that alerts or records events not in accordance with prescribed medical care instructions. The temperature strip 166 may be connected to a control unit 162 (described below) by a wire or cable.

In some embodiments, the lower component 115 may be constructed from or include a translucent structural scaffolding component to support vacuum control of the suction element 136 and light transmission from the LED layer 123. In some embodiments, the scaffolding structure may be coated with a medication and/or an antibacterial element. In some embodiments, the lower component 115 may be constructed from a material that is antimicrobial. In some embodiments, the peripheral material 139 may have a silver ion coating structure applied thereto that may facilitate closing fractures at the skin 4 or wound site 6 and produce and anti-bacterial environment to promote tissue healing. In some embodiments, the lower component 115 may include a conductive suture on the peripheral material 139 that interfaces with the skin 4 of a subject 2 and allows for electrical energy to be carried to and in proximity to the wound site 6. The lower component 115 may include conductive electrodes integrated into the lower component 115 that enable delivery of electrical stimulation to the wound site 6 for purposes or reducing pain and inflammation at the wound site 6. In some embodiments, the conductive electrodes are provided around the periphery of the peripheral material 139.

The dressing system 110 comprises a cooling chamber 124 external to the dressing 112. The cooling chamber 124 may be disposed within a cooling chamber body 125, the body 125 constructed of any suitable material. In some embodiments, a thermoelectric unit 168, such as a thermoelectric cooler or a heat exchanger, may be used to change the temperature of the liquid within the chamber 124 in order to cool the liquid to a desired temperature.

The dressing system 110 may include a control unit 162. The control unit 162 (along with pumps 130, 142 and 164) may be disposed on or within any suitable component of the system 110, such as within the cooling chamber body 125. The control unit 162 may contain a user interface having various controls and settings displayed for modification and operation by a user, including but not limited to, power (e.g., on and off), temperature (e.g., on, off, set, and maintain), vacuum (e.g., on, off, set, and automatic off when maximum threshold of reservoir(s) is reached), and medication delivery (e.g., on, off, deliver medication, or extract substances). The control unit 162 may also include status indicators for the various controls and settings, including but not limited to, the power battery status, charging status, lower power warning, vacuum setting, medication volume status, reservoir(s) volume status, and temperature status.

The control unit 162 may cause the pump(s) 130, 142, 164 to operate when certain controls and/or settings are met or selected by a user. By way of example, if the initial threshold temperature is 90 degrees F., once the control unit 162 reads the temperature being above 90 degrees, F, the control unit 162 could cause the pump 130 to operate, thereby circulating the liquid and cooling the skin 4. Once the temperature strip 166 reads a certain subsequent threshold temperature, such as 40 degrees F., the control unit 162 may cause the turn off the pump 130. The control unit 162 may cause the pump 130 to operate at variable speeds based on one or more threshold temperatures (e.g., a low pump speed at a cold threshold temperature, a moderate pump speed at a warm threshold temperature, a high pump speed at a hot threshold temperature). The control unit 162 may communicate with the pump 130 and the temperature strip 166 wiredly or wirelessly. The control unit may operatively and selectively control the thermoelectric unit 168. The external power source 181 may power the pumps 130, 142, 164, the control unit 162, and the thermoelectric unit 168.

The dressing system 110 may comprise a body 143 external to the dressing 112. The body 143 may be, for example, disposed on a medical cart 144 (e.g., a medical stand), located proximate to the subject 2. One or more of the cooling chamber 124, the drain cavity 138, or the pump(s) 130, 142, 164 may be disposed on or in the body 143. The body 143 may be constructed of any suitable material, such as metal or a polymer (whether rigid or flexible). The body 143 being external to the dressing 112 is particularly advantageous, as it enables the dressing 112 to be compact and comfortable for the subject 2 to wear.

In some embodiments, the dressing 112 may be dimensioned and shaped to be cooperatively received on a face of the subject 2. The suction element 136 may be disposed on or in the lower component 115. Advantageously, when the dressing 112 (using suction of the suction element 136) is used in the face of the subject 2, the dressing 112 promotes epidermal and dermal health and rejuvenation (e.g., a youthful appearance).

The dressing 112 may have a low profile design and be constructed of a soft and pliable material. This design is especially advantageous in facial applications, as eliminating or reducing sharp folds and having a profile that is shaped to contour a face prevents skin irritation. Using the features described herein, the dressing system 110 may be varied to include a dressing 112 that has a site-specific profile for use in, for example, plastic surgery, neurosurgery, urological surgery, ENT surgery, maxillofacial surgery, orthopedic surgery, and general surgery. That is, the dressing 112 may have a cooperative profile with the site-specific area on the subject 2 that is desired to be cooled and/or suctioned (e.g., neck, top or crown of head, nose, etc.) such that the dressing 112 can be worn on the subject 2 without folds or wrinkles in the dressing 112. This site-specific profile feature is believed to add to the healing and rejuvenating attributes of the dressing system 110.

In some embodiments, the dressing system 110 may incorporate ultrasonics technology into the dressing 112. The upper component 114 may include one or more wires or cables (not shown) that enable the delivery or transmission of electrotherapy and/or ultrasound therapy.

Embodiments of the dressing system 110 described herein may be used in methods of promoting healing in a subject, such as promoting healing of the skin 4 of the subject 2. The skin 4 of the subject 2 may comprise a wound site 6, facial skin 4, or both. The method may include applying negative pressure at, or proximate to, the dressing 112, such as via suction element 136. The method may include suctioning and capturing wound drainage or sweat. Also advantageously, the cooling and/or suction features of the present dressing system 100 reduce, or eliminate, postoperative pain. Also advantageously, the LED feature of the present invention may promote cellular efficiency of the skin 4 or wound of a subject 2, and/or reduce inflammation and pain.

Although embodiments of the disclosure have been described using specific terms, devices, and methods, such description is for illustrative purposes only. The words are words of description rather than of limitation. It is to be understood that changes and variations may be made by those of ordinary skill in the art without departing from the spirit or scope of the present disclosure, which is set forth in the following claims. It is further noted that any range provided herein provides support and a basis for any subset within that range. Further embodiments of the disclosure contain combinations, or exclusions, of different embodiments described herein.

Thus, although there have been described particular embodiments of the present invention of a new and useful dressing system, it is not intended that such references be construed as limitations upon the scope of this invention except as set forth in the following claims.

What is claimed is:

1. A dressing system, comprising:
a dressing including a reservoir and at least one light-emitting diode;
a cooling chamber external to the dressing;
a pump configured to circulate a cooled liquid between the cooling chamber and the reservoir of the dressing; and
a cooled liquid hose for circulating the cooled liquid between the cooling chamber and the reservoir of the dressing.

2. The dressing system of claim 1, wherein the at least one light-emitting diode emits light at a wavelength approximately between 630 and 830 nanometers.

3. The dressing system of claim 1, wherein the at least one light-emitting diode emits light at a wavelength approximately between 405 and 470 nanometers.

4. The dressing system of claim 1, wherein the at least one light-emitting diode emits a range of wavelengths.

5. The dressing system of claim 1, wherein the dressing comprises an upper component including a first coupling element and a lower component including a second coupling element, wherein the first coupling element and the second coupling element are configured to removably connect the upper component to the lower component.

6. The dressing system of claim 1, further comprising a suction element configured to remove bodily fluids from an area proximate to the dressing.

7. The dressing system of claim 6, further comprising a drain cavity for receiving bodily fluids and a drain hose, wherein the drain hose is in fluid communication with the suction element.

8. The dressing system of claim 1, further comprising a flush port element configured to deliver and extract a substance to an area proximate the dressing.

9. A dressing system, comprising:
a dressing including an upper component and a lower component, wherein the upper component includes a reservoir and at least one light-emitting diode;
a cooling chamber external to the dressing;
a pump configured to circulate a cooled liquid between the cooling chamber and the reservoir of the dressing; and
a cooled liquid hose for circulating the cooled liquid between the cooling chamber and the reservoir of the dressing.

10. The dressing system of claim 9, wherein the at least one light-emitting diode emits light at a wavelength between 630 and 830 nanometers.

11. The dressing system of claim 9, wherein the at least one light-emitting diode emits light at a wavelength between 405 and 470 nanometers.

12. The dressing system of claim 9, wherein the upper component and the lower component are selectively detachable.

13. The dressing system of claim 9, wherein the upper component comprises a first coupling component provided around the periphery thereof and the lower component comprises a second coupling component provided around the periphery thereof, wherein the first and second coupling components are selectively connectable to one another.

14. The dressing system of claim 9, wherein the lower component further comprises a suction element configured to remove bodily fluids from the area proximate to the dressing.

15. The dressing system of claim 14, further comprising a drain cavity for receiving bodily fluids and a drain hose, wherein the drain hose is in fluid communication with the suction element.

16. A dressing system, comprising:
an upper dressing component comprising:
a reservoir layer configured for retaining a fluid therein;
a light-emitting diode layer comprising a plurality of light-emitting diodes arranged across a surface of the upper dressing component; and
a first coupling component provided around at least a portion of a perimeter edge of the upper dressing component;
a lower dressing component comprising:
a suction element layer, the suction element layer including a suction element for providing negative pressure at the lower dressing component and a drain cavity;
a peripheral material layer positioned below the suction element layer; and
a second coupling component provided around at least a portion of a perimeter edge of the lower dressing component, wherein the second coupling element is selectively connectable to the first coupling element of the upper dressing component, wherein the upper dressing component and the lower dressing component collectively form a dressing;
a cooling chamber external to the dressing;
a cooled liquid hose for circulating a cooled liquid between the cooling chamber and the reservoir of the dressing;
a drain hose for moving material suctioned from the suction element to the drain cavity, wherein the material is kept separate from the cooled liquid;
a pump configured to circulate the cooled liquid between the cooling chamber and the reservoir of the dressing and configured to create suction to the suction element to the drain hose; and
a pump valve for modulating suction to the drain hose and to the cooling hose.

17. The dressing system of claim 16, wherein the upper component and the lower component are selectively detachable.

18. The dressing system of claim 16, wherein the plurality of light-emitting diodes emit light at a wavelength between 630 and 830 nanometers.

19. The dressing system of claim 16, wherein the plurality of light-emitting diodes emit light at a wavelength between 405 and 470 nanometers.

20. The dressing of claim 16, further comprising a temperature strip provided on the lower component of the dressing and configured for determining a temperature at a surface of the peripheral material.
